(19) United States
(12) Patent Application Publication
(10) Pub. No.: US 2003/0073828 A1
(43) Pub. Date: Apr. 17, 2003

(54) Epimerase gene and use thereof

(75) Inventors: Ziyu Dai, Richland, WA (US); LiFang Shi, Richland, WA (US); Brian S. Hooker, Kennewick, WA (US)

Correspondence Address: FOLEY AND LARDNER, SUITE 500, 3000 K STREET NW, WASHINGTON, DC, 20007, US

(73) Assignee: Battelle Memorial Institute

(21) Appl. No.: 10/060,275

(22) Filed: Feb. 1, 2002

Related U.S. Application Data
(60) Provisional application No. 60/265,311, filed on Feb. 1, 2001.

(51) Int. Cl. C07H 21/02; C07H 21/04

(52) U.S. Cl. 536/23.1

(57) ABSTRACT

This invention provides transgenic plants that have increased or decreased activity of uridine 5′-diphospho-galactose 4-epimerase. Controlling the level of uridine 5′-diphospho-galactose 4-epimerase in the transgenic plants is used to regulate the nutritional profile of the plant by controlling the use of glucose and/or galactose. An isolated polynucleotide coding for the potato uridine 5′-diphospho-galactose 4-epimerase protein, its antisense equivalent and the nucleic acid sequence of potato uridine 5′-diphospho-galactose 4-epimerase are also provided. The present invention also provides a method for reducing the activity of uridine 5′-diphospho-galactose 4-epimerase activity in plants. The present invention also provides a method of producing bulk quantities of uridine 5′-diphospho-galactose 4-epimerase enzyme in transgenic plants.

### BACKGROUND OF THE INVENTION

1. Field of the Invention

The present invention relates to a cloned gene coding for an enzyme involved in the conversion of sugars in potato. More particularly this invention relates to a cloned gene encoding an epimerase which catalyzes the interconversion of UDP-glucose and UDP-galactose.

2. Description of the Related Art

Carbohydrates are the preferred dietary source of energy in human nutrition because they generally comprise the main energy source available from foodstuffs. In poorer countries, carbohydrates provide about 85% of total food intake. Carbohydrates also remain important in industrial applications and consumer products and accordingly have significant commercial value. The primary source for carbohydrates around the world is crop plants. Many of the improvements in crop plants over time have focused on altering the amounts and ratios of carbohydrates produced by these plants.

Manipulating the carbohydrate production and ratios in plants, however, requires a delicate balance because polysaccharides are the major product for storing energy in most plants. Most commonly, long chain polymers of glucose, referred to as glucans, are used by plants in energy storage. Starch is the predominant energy storing glucan utilized by higher plants and is a major source of caloric intake in humans and animals and is also important in the production of other chemicals, for example ethanol. Furthermore, the level of polysaccharides present in plants constitute a key component in determining the desirability of using plants in food and industrial uses because carbohydrates affect the characteristics of the final product, including taste, texture, nutritional profile and the like. For example, the starch in potatoes which are stored at cold temperatures breaks down into sugars and renders these sweetened potatoes unacceptable for use in processed products such as potato chips and french fries.

Biosynthesis of glucan molecules occurs through the basic building block intermediate of an activated glucose molecule. Activation of glucose occurs when a glucose molecule is conjugated to a nucleoside diphosphate, for example uridine diphosphate (UDP). Activated glucose molecules are then incorporated into polysaccharides by synthase enzymes. Activated glucose molecules, and in particular UDP-glucose, are also important in synthesizing disaccharides and complex sugars.

Another important carbon source for plants is galactose. Because glucose and galactose are isomers, glucose can be converted to galactose and vice versa through an enzymatic pathway. Galactose is an important constituent of galactolipids, cell-wall polysaccharides, glycoproteins and transport metabolites. Galactolipids also make up roughly 75% of the polar lipids found in chloroplasts. Accordingly, both glucose and galactose play crucial roles in plant development and survival, albeit through different mechanisms, and the control of the utilization of glucose and galactose in plants can vastly increase their commercial value. Likewise, the reversible interconversion of UDP-glucose and UDP-galactose by 5′ diphospho-galactose 4-epimerase (DGE) is an important point of control in plant carbohydrate metabolism.

There is a need, therefore, for isolated DNA molecules coding for DGE that can be used to regulate carbohydrate metabolism in transgenic plants. Also required is a method for producing DGE in bulk from a transgenic plant.

### SUMMARY OF THE INVENTION

It is therefore an object of the present invention to provide an isolated DGE nucleotide that codes for DGE corresponding to polynucleotides that code for DGE. Other polynucleotides of the present invention provide antisense sequences which are used to regulate epimerase activity in plants. The polynucleotide of the present invention can be DNA or cDNA. Also provided is a vector, a plasmid, a host cell, and a plant cell comprising the polynucleotide molecule of the present invention. Particularly provided are plants containing the polynucleotide molecule of the present invention that are crop plants and especially those crop plants which store starch as energy reserves, e.g. potatoes, Brassica, maize, rice and wheat. The polynucleotide molecules of the present invention can be operably linked to a promoter that controls the expression of the polynucleotide molecules. The promoter linked to the polynucleotide molecules can be constitutive, inducible or developmentally regulated.

Further provided is a method for reducing the activity of DGE in plants comprising a) preparing an antisense polynucleotide molecule to a polynucleotide molecule that codes for DGE; b) transforming a recipient plant cell with the polynucleotide molecule; c) regenerating a plant from the recipient cell which has been transformed with the polynucleotide molecule; and d) identifying a fertile transgenic plant comprising the polynucleotide molecule exhibiting reduced DGE activity. The method can further comprise selfing the fertile transgenic plant to obtain a transgenic plant that is homozygous for the polynucleotide molecule.

The present invention also provides a method for producing DGE enzyme in a host cell to provide a source of the enzyme. DGE enzyme is purified in bulk from the transformed cell or plant derived therefrom.

### BRIEF DESCRIPTION OF THE DRAWINGS

FIG. 1 shows the DNA sequence of a gene coding for uridine 5′-diphospho-galactose 4-epimerase (dge) isolated from potato. The amino acid sequence of the DGE enzyme coded for by this gene is shown above the polynucleotide sequence.

FIG. 2 shows the results of a Northern blot analysis for dge in potato plants incubated in light and dark.

FIG. 3 is the polynucleotide sequence or an antisense gene for the dge gene of FIG. 1.

### DETAILED DESCRIPTION OF THE PREFERRED EMBODIMENT

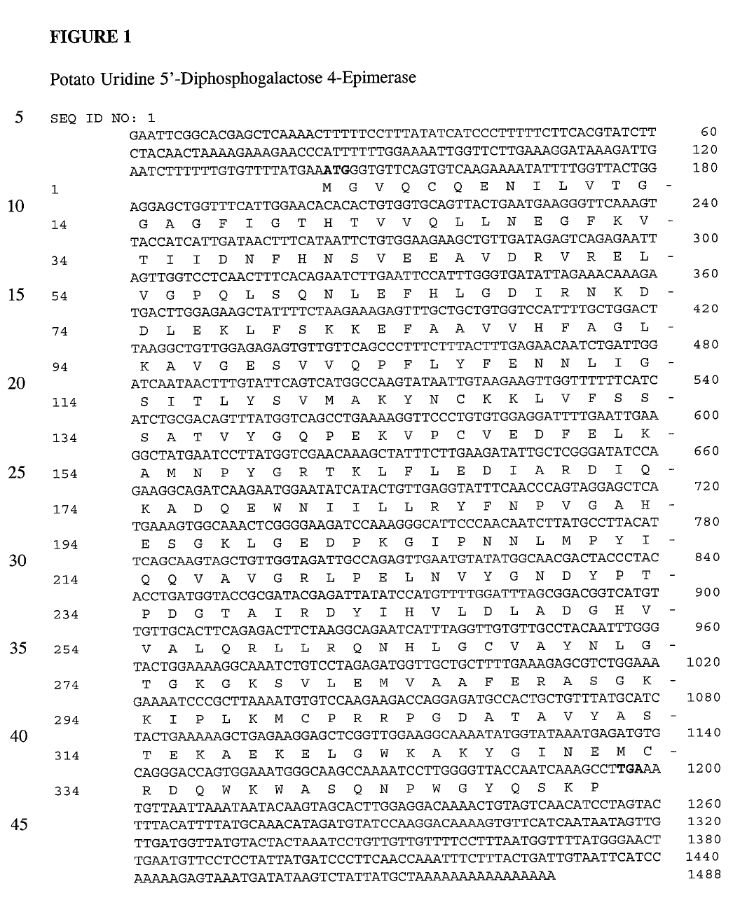

A preferred embodiment of the present invention utilizes the polynucleotide sequence and amino acid sequence set forth in FIG. 1. The dge gene isolated from potato has been named psen-1 and therefore the polynucleotide sequence of psen-1 as shown in FIG. 1. As described above, the enzymes in the DGE group catalyze the reversible interconversion of UDP-glucose and UDP-galactose. The skilled artisan will recognize that structure ultimately defines function, and that variants bearing the closest structural relationship to the molecules of FIG. 1 are most likely to preserve their biological function. In this case, a “functional” epimerase protein has the ability to reversibly interconvert UDP-glucose and UDP-galactose. A functional polynucleotide is defined as any polynucleotide that codes for an enzyme that has DGE activity. Furthermore, a functional polynucleotide is defined herein as a polynucleotide that hybridizes under stringent conditions to a polynucleotide comprising the DNA sequence set forth in FIG. 1 and codes for an enzyme having DGE activity.

1. Definitions

As used herein, the term gene should be understood to be a full-length DNA sequence encoding a protein or an RNA molecule, as well as a truncated fragment thereof. A gene can be naturally occurring or synthetic.

Marker gene should be understood as a gene coding for a selectable marker (e.g., encoding antibiotic or herbicide resistance) or a screenable marker (e.g., coding for a gene product which permits detection or transformed cells or plants). The marker gene for the polynucleotide molecule of the present invention can be any nucleotide sequence that codes for a protein or polypeptide which allows transformed cells to be distinguished from non-transformed cells. The marker gene can be, for example, a herbicide resistance gene, an antibiotic resistance gene, a β-glucuronidase (GUS) gene, or a luciferase gene.

A promoter is a nucleotide sequence upstream from the transcriptional initiation site and which contains all the regulatory regions required for transcription. Typically, a promoter is located in the 5′ region of a gene, proximal to the transcriptional start site. Examples of promoters suitable for use in DNA constructs of the present invention include viral, fungal, bacterial, animal and plant-derived promoters capable of functioning in plant cells. The promoter can be selected from so-called constitutive promoters or inducible promoters. If a promoter is an inducible promoter, then the rate of transcription increases in response to an inducing agent. In contrast, the rate of transcription is not regulated or largely unregulated by an inducing agent, if the promoter is a constitutive promoter. Examples of suitable inducible or developmentally regulated promoters include the napin storage protein gene (induced during seed development), the malate synthase gene (induced during seedling germination), the small sub-unit RUBISCO gene (induced in photosynthetic tissue in response to light), the patatin gene (highly expressed in potato tubers) and the like. Examples of suitable constitutive promoters include the cauliflower mosaic virus 35S (CaMV 35S) and 19S (CaMV 19S) promoters, the nopaline synthase promoter, octopine synthase promoter, and the like. Promoters can also be developmentally regulated promoters. It will be appreciated that the promoter employed in the present invention should be strong enough to control the transcription of a sufficient amount of the epimerase encoded by the polynucleotide sequence to provide sufficient interconversion of glucose and galactose to give a plant the desired carbohydrate profile.

A tissue-preferred promoter is a DNA sequence that, when operably linked to a gene, directs a higher level of transcription of that gene in a specific tissue than in some or all other tissues in an organism. Examples of such promoters are a stem-specific promoter such as the AdoMet-synthetase promoter (Peleman et al., 1989, The Plant Cell 1:81-93), a tuber-specific promoter (Rocha-Sosa et al., 1989, EMBO J. 8:23-29). For example, a seed-preferred promoter is a DNA sequence that directs a higher level of transcription of an associated gene in plant seeds. Examples of seed-preferred promoters include the seed specific promoter of the USP gene of Vicia faber (U.S. Pat. No. 5,917,127); the 7S protein promoter of soybean (Bray et al., 1987, Planta 172:364-370) and the 2S promoter (Krebbers et al., 1988, Plant Physiol. 87:859-866).

A terminator is a DNA sequence at the 3′-end of a transcribed unit which signals termination of transcription. These elements are 3′-non-transcribed sequences containing polyadenylation signals which act to cause the addition of polyadenylate sequences to the 3′ end of primary transcripts. Examples of terminators particularly suitable for use in nucleotide sequences and DNA constructs of the invention include the nopaline synthase polyadenylation signal of Agrobacterium tumefaciens, the 35S polyadenylation signal of CaMV, octopine synthase polyadenylation signal and the zein polyadenylation signal from Zea cans.

An isolated nucleic acid molecule is a fragment of a nucleic acid molecule that has been separated from the nucleic acid of an organism or other natural environment of the nucleic acid. An isolated nucleic acid molecule includes a chemically-synthesized nucleic acid molecule. Other examples of isolated nucleic acid molecules include in vivo or in vitro transcripts of the nucleic acids of the present invention.

Isolated polypeptides are polypeptides not in their naturally occurring form or have been purified to remove at least some portion of cellular or non-cellular molecules with which the proteins are naturally associated. However, the “isolated” protein can be included in compositions containing other polypeptides for specific purposes, for example, as stabilizers, where the other polypeptides can occur naturally associated with at least one polypeptide of the present invention.

The terms “complementary” or “complementarity” refer to the capacity of purine and pyrimidine nucleotides to associate non-covalently to form partial or complete double stranded nucleic acid molecules. The following base pairs are naturally complementary: guanine (G) and cytosine (C); adenine (A) and thymine (T); and adenine (A) and uracil (U).

Complementary DNA (cDNA) is a single-stranded DNA molecule that is formed from a mRNA template by the enzyme reverse transcriptase. Typically, a primer complementary to portions of an mRNA is employed for the initiation of reverse transcription. Those skilled in the art also use the term “cDNA” to refer to a double-stranded DNA molecule consisting of such a single-stranded DNA molecule and its complementary DNA strand.

The term expression refers to the biosynthesis of a gene product. For example, in the case of a structural gene, expression involves transcription of the structural gene into mRNA and the translation of mRNA into one or more polypeptides. In the case of an antisense gene, expression involves transcription of the antisense DNA into an antisense RNA molecule that is complementary to the sense mRNA.

In eukaryotes, RNA polymerase II catalyzes the transcription of a structural gene to produce mRNA. A DNA molecule can be designed to contain an RNA polymerase II template in which the RNA transcript has a sequence that is complementary to that of a specific mRNA. The RNA transcript is termed an antisense RNA and a DNA sequence that codes for the antisense RNA is termed an antisense gene. An antisense RNA molecule inhibits the expression of the gene to which it corresponds.

A vector is a DNA molecule, such as a plasmid, cosmid, viruses or bacteriophage, that has the capability of replicating autonomously in a host cell. Cloning vectors typically contain a marker gene and one or a small number of restriction endonuclease recognition sites for insertion of foreign DNA sequences without affecting the essential biological function of the vector.

An expression vector is a DNA molecule comprising a gene that is expressed in a host cell. Typically, gene expression is placed under the control of certain regulatory elements, including, for example, constitutive or inducible promoters, tissue-preferred regulatory elements, and enhancers. Such a gene is said to be “operably linked to” or “operatively linked to” the regulatory elements.

“Host cell” refers to any eukaryotic, prokaryotic, or other cell that is suitable for propagating or expressing an isolated nucleic acid that is introduced into the cell by any suitable means known in the art. The cell can be part of a tissue or organism, isolated in culture or in any other suitable form. A recombinant host can be any prokaryotic or eukaryotic cell that contains either a cloning vector or expression vector. This term also includes those prokaryotic or eukaryotic cells that have been genetically engineered to contain an isolated gene in the chromosome or genome of the host cell. Host cells according to the present invention comprising a polynucleotide molecule of the present invention or a vector containing a polynucleotide of the present invention can be cultivated under conditions which produce a polypeptide having epimerase activity. The polypeptide can then be purified.

A transgenic plant is a plant having one or more plant cells that contain a foreign gene. The foreign gene is usually non-native, meaning that it is originated from a source other than the host plant and does not share sequence homology to the host genome. The foreign gene can also be native, meaning that it has the nucleotide sequence found in the host. The transgenic plant is made by one of many transformation methods well known in the art. As used herein, a fertile transgenic plant is capable of transmitting a foreign gene to its progeny of further descendants. As used herein, the term transformation refers to alteration of the genotype of a host plant by the introduction of native or non-native nucleic acid sequences into the genomes of the plant cell.

The term isoforms refer to genetic variants of a polynucleotide which, either share the same regulatory function, if the sequence of the polynucleotide spans the regulatory region, or code for protein isoforms with the same function, if the sequence of the polynucleotide covers the coding region. Protein isoforms refer to a set of protein molecules which have the same physical and physiological properties and the same biological function, and whose amino acid sequences have several amino acid differences.

The term operably linked is used to describe the connection between regulatory elements and a gene or its coding region. That is, gene expression is typically placed under the control of certain regulatory elements, including constitutive or inducible promoters, tissue-specific regulatory elements, and enhancers. Such a gene is said to be “operably linked to” or “operatively linked to” the regulatory elements.

Sequence homology is used to describe the sequence relationships between two or more nucleic acids, polynucleotides, proteins, or polypeptides, and is understood in the context and in conjunction with the terms including: (a) reference sequence, (b) comparison window, (c) sequence identity, (d) percentage of sequence identity, and (e) substantial identity or “homologous.”

(a) A reference sequence is a defined sequence used as a basis for sequence comparison. A reference sequence can be a subset or the entirety of a specified sequence; for example, as a segment of a full-length cDNA or gene sequence, or the complete cDNA or gene sequence.

(b) A comparison window includes reference to a contiguous and specified segment of a polynucleotide sequence, wherein the polynucleotide sequence can be compared to a reference sequence and wherein the portion of the polynucleotide sequence in the comparison window can comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Optimal alignment of sequences for comparison can be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997).

As those of ordinary skill in the art will understand, BLAST searches assume that proteins can be modeled as random sequences. However, many real proteins comprise regions of nonrandom sequences which can be homopolymeric tracts, short-period repeats, or regions enriched in one or more amino acids. Such low-complexity regions can be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) Sequence identity or identity in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity can be adjusted upwards to correct for the conservative nature of the substitution. Sequences which differ by such conservative substitutions are said to have sequence similarity or similarity. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The term “substantial identity” or “homologous” means that a polynucleotide comprises a sequence that has at least 75% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins coded for by two nucleotide sequences by taking into account codon degeneracy, amino acid similarity, reading frame positioning and the like. Substantial identity of amino acid sequences for these purposes normally means sequence identity of at least 85%, preferably at least 90%, more preferably at least 95%, and most preferably at least 97%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This can occur, e.g., when a copy of a nucleic acid is created using the maximum codon degeneracy permitted by the genetic code. One indication that two nucleic acid sequences are substantially identical is that the polypeptide which the first nucleic acid has a similar enzyme activity as the polypeptide encoded by the second nucleic acid.

(e) (ii) The terms “substantial identity” or “homologous” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch, J. Mol. Biol. 48: 443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions which are not identical can differ by conservative amino acid changes.

Nucleic Acid variants within the invention also can be described by reference to their physical properties in hybridization. One skilled in the field will recognize that nucleic acid can be used to identify its complement or homologue, using nucleic acid hybridization techniques. It will also be recognized that hybridization can occur with less than 100% complementarity. However, given appropriate choice of conditions, hybridization techniques can be used to differentiate among DNA sequences based on their structural relatedness to a particular probe. For guidance regarding such conditions see, for example, Sambrook et al., 1989, Molecular Cloning—A Laboratory Manual, 2nd ed., Vol. 1-3; and Ausubel et al., 1989, Current Protocols in Molecular Biology, Green Publishing Associates and Wiley Interscience, N.Y.

Structural relatedness between two polynucleotide sequences can be expressed as a function of “stringency” of the conditions under which the two sequences will hybridize with one another. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences (Bolton et al., 1962, Proc. Natl. Acad. Sci. 48:1390.Hybridization stringency is thus a function of many factors, including overall DNA concentration, ionic strength, temperature, probe size and the presence of agents that disrupt hydrogen bonding. Factors promoting hybridization include high DNA concentrations, high ionic strengths, low temperatures, longer probe size and the absence of agents that disrupt hydrogen bonding.

Hybridization usually is done in two stages. First, in the “binding” stage, the probe is bound to the target under conditions favoring hybridization. A representative hybridization solution comprises 6× SSC, 0.5% SDS, 5× Denhardt's solution and 100 μg of non-specific carrier DNA. See Ausubel et al., supra, section 2.9, supplement 27 (1994). A stock 20× SSC solution contains 3M sodium chloride, 0.3M sodium citrate, pH 7.0. Of course many different, yet functionally equivalent, buffer conditions are known. For high stringency, the temperature is between about 65° C. and 70° C. in a hybridization solution of 6× SSC, 0.5% SDS, 5× Denhardt's solution and 100 μg of non-specific carrier DNA. Moderate stringency is between at least about 40° C. to less than about 65° C. in the same hybridization solution. In both cases, the preferred probe is 100 bases selected from contiguous bases of the polynucleotide sequence set forth in SEQ ID NO: 1.

Second, the excess probe is removed by washing, which is most important in determining relatedness via hybridization. Washing solutions typically contain lower salt concentrations. A medium stringency wash solution contains the equivalent in ionic strength of 2× SSC and 0.5-0.1% SDS. A high stringency wash solution contains the equivalent in ionic strength of less than about 0.2× SSC and 0.1% SDS, with a preferred stringent solution containing about 0.1× SSC and 0.1% SDS. The temperatures associated with various stringencies are the same as discussed above for “binding.” The washing solution also typically is replaced a number of times during washing. For example, typical high stringency washing conditions comprise washing with 2× SSC plus 0.05% SDS five times at room temperature, and then washing with 0.1× SSC plus 0.1% SDS at 68° C. for 1 h. Blots containing the hybridized, labeled probe are exposed to film for one to three days.

The present invention includes nucleic acid molecules that hybridize to the molecules of FIG. 1 under high stringency binding and washing conditions. Preferred molecules are those that are at least 50% of the length of any one of those depicted in FIG. 1. Particularly preferred molecules are at least 75%, more preferably 85%, most preferably 95% of the length of those molecules.

Structural variants can also be due to substitutions, insertions, additions, and deletions. With regard to amino acid sequence, “substitutions” generally refer to alterations in the amino acid sequence that do not change the overall length of the polypeptide, but only alter one or more amino acid residues, substituting one for another in the common sense of the word. “Insertions,” unlike substitutions, alter the overall length of the polypeptide. Insertions add extra amino acids to the interior (not the amino- or carboxyl-terminal ends) of the subject polypeptide. “Deletions” diminish the overall size of the polypeptide by removal of amino acids from the interior or either end of the polypeptide. Preferred deletions remove less than about 30% of the size of the subject molecule. “Additions,” like insertions, also add to the overall size of the protein. However, instead of being made within the molecule, they are made on the N- or C-terminus of the encoded protein. Unlike deletions, additions are not very size-dependent. Indeed, additions can be of virtually any size. Preferred additions, however, do not exceed about 100% of the size of the native molecule. The artisan understands “additions” also to encompass adducts to the amino acids of the native molecule.

2. Promoter Constructs, Promoter-Enhancer Combinations

The polynucleotide coding for an epimerase of the present invention can be used in connection with an external enhancer element to achieve a high-level of gene expression, as well as to enable the high-level control of the enhanced expression.

An enhancer element is cis-acting and is generally upstream from, and within 5000 bp of, a promoter. The enhancer element is preferably located within about 2000 bp, most preferably adjacent to, or within about 1000 bp of, the transcription initiation codon of the promoter. Conventionally, the initial nucleotide of the transcribed mRNA is designated +1, thus the sequence containing the enhancer is preferably located upstream from about −50 to about −1000 bp, usually from −50 to about −800, and more specifically from −50 to −500 bp from the transcription initiation codon. The enhancer element can be located upstream or downstream in relation to the promoter it affects. Alternatively, the enhancer element can be positioned within introns in a transcription unit.

The external enhancer elements that can be used in conjunction with a promoter to control expression of the epimerase of the present invention are themselves separately functional; each individually, in tandem, or dispersed, is independently capable of affecting gene transcription of a promoter operatively linked thereto. In one preferred embodiment, the promoter and the external enhancer elements can be variously combined to provide synergistic effect in increasing the gene transcription capabilities of the polynucleotide sequence of the present invention operatively linked to these elements. In a further embodiment, the promoter and the external enhancer elements can be variously combined to confer regulatable control to an operably linked gene of the present invention.

Preferably, an isolated polynucleotide of the present invention is operatively linked to an external enhancer and the suitable enhancer can be any plant-compatible enhancer. The overall transcriptional activity of the dge gene can be increased or otherwise modified by use of particular combinations of promoter and enhancer. The expression of structural genes employed in the present invention therefore can be operably linked to the promoter-enhancer combinations according to the present invention. The recombinant constructs designed as such can be modified, if desired, to affect their control characteristics.

Environmental factors and hormonal agents can be utilized to test the activities of the nucleic constructs according to the present invention, and thus to identify the responsive promoter constructs for various conditions. The transcriptional activities can be determined by measuring the levels of expression of a dge, such as psen-1 of the present invention, fused in frame to a reporter gene. For example, psen-1 is fused in frame to the well known screenable marker gus and GUS activity is assayed.

3. Construction of Nucleic Acids

The isolated nucleic acids of the present invention can be made using (a) standard recombinant methods, (b) synthetic techniques, (c) purification techniques, or combinations thereof, as well known in the art. The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. Additional sequences that can be inserted include adapters or linkers for cloning and/or expression. Use of cloning vectors, expression vectors, adapters, and linkers is well known in the art.

The various restriction enzymes disclosed and described herein are commercially and/or available and the manner of use of the enzymes including reaction conditions, cofactors, and other requirements for activity are well known to one of ordinary skill in the art (New England BioLabs, Boston; Life Technologies, Rockville, Md.). Reaction conditions for particular enzymes are preferably carried out according to the manufacturer's recommendation.

A variety of cross-linking agents, alkylating agents and radical generating species as pendant groups on polynucleotides of the present invention can be used to bind, label, detect, and/or cleave nucleic acids using methods and reagents known in the art.

i) Recombinant Methods for Constructing Nucleic Acids

The isolated nucleic acid compositions of this invention can be obtained from biological sources using any number of cloning methodologies known to those of skill in the art. Oligonucleotide probes that selectively hybridize to the polynucleotides of the present invention can be used to identify the desired sequence in a cDNA or genomic DNA library. Isolation of RNA and construction of cDNA and genomic libraries is well known to those of ordinary skill in the art.

ii) Synthetic Methods for Constructing Nucleic Acids

The isolated nucleic acids of the present invention can also be prepared by direct chemical synthesis using the solid phase phosphoramidite triester method (Beaucage and Caruthers, Tetra. Letts. 22(20): 1859-1862 (1981)); an automated synthesizer (VanDevanter et al., Nucleic Acids Res., 12: 6159-6168 (1984)); or the solid support method of U.S. Pat. No. 4,458,066. Chemical synthesis generally produces a single stranded oligonucleotide. This can be converted into double stranded DNA by hybridization with a complementary sequence, or by polymerization with a DNA polymerase using the single strand as a template. One of skill will recognize that while chemical synthesis of DNA is limited to sequences of about 100 bases. Longer sequences can be obtained by the ligation of shorter sequences.

iii) Recombinant Expression Cassettes

The present invention further provides recombinant expression cassettes comprising a nucleic acid of the present invention, and operably linked to transcriptional initiation regulatory sequences that will direct the transcription of the polynucleotide in the intended host cell. Both heterologous and endogenous promoters can be employed to direct expression.

In some embodiments, isolated nucleic acids which serve as promoter or enhancer elements can be introduced in the appropriate position (generally upstream) of a non-heterologous form of a polynucleotide of the present invention so as to up, or down regulate, expression of a polynucleotide of the present invention. For example, endogenous promoters can be altered in vivo by mutation, deletion, and/or substitution. Suitable promoters include the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

The polynucleotides can optionally be joined to a vector containing a selectable marker for propagation in a host. Such markers include, e.g., bialaphose or neomycin resistance for eukaryotic cell culture and tetracycline or ampicillin resistance genes for culturing in Escherichia coli and other bacteria. Representative examples of appropriate hosts include, but are not limited to, bacterial cells, such as E. coli, Streptomyces and Salmonella typhimurium cells; and fungal cells, such as yeast cells. One skilled in the art will also recognize that dge, such as psen-1, can also serve as a suitable marker gene under appropriate regulation of the gene in host cells grown on galactose or glucose containing medium.

4. Control of In Planta Epimerase Activity

The present invention discloses a method to reduce or eliminate the epimerase activity in a plant. Controlling the DGE activity in planta can alter the energy storage levels (through starch manufacture or depletion) present in a plant, and particularly in crop plants. Dormann and Benning, The Plant Journal 13(5): 641-652 (1998). Any well known method for reducing or eliminating gene expression can be used, such as antisense technology, cosuppression, transcriptional silencing, and ribozymes. Cosuppression is the phenomenon in which the expression of homologous genes (including endogenous copies) is suppressed by the introduction of multiple insertions of related transgenes.

Antisense technology is a versatile approach for shutting-off endogenous cellular genes and extinguishing cellular gene expression. The principle is to produce in a cell an RNA or single-stranded DNA molecule complementary to the mRNA of the target gene. Although the mechanism of action of antisense technology is not completely understood, the antisense molecule may base-pair with the cellular mRNA preventing its translation. The protocol was originally developed for the control of the gene encoding polygalacturonase during fruit ripening in tomato. See, for example, Smith et al., Nature 334:724-726 (1988). Considerable effort has been devoted to the development of antisense RNA technology for the production of novel plant mutants which have the advantage of being stably inherited. Schuch, Soc. Exp. Biol. 117-127 (1991).

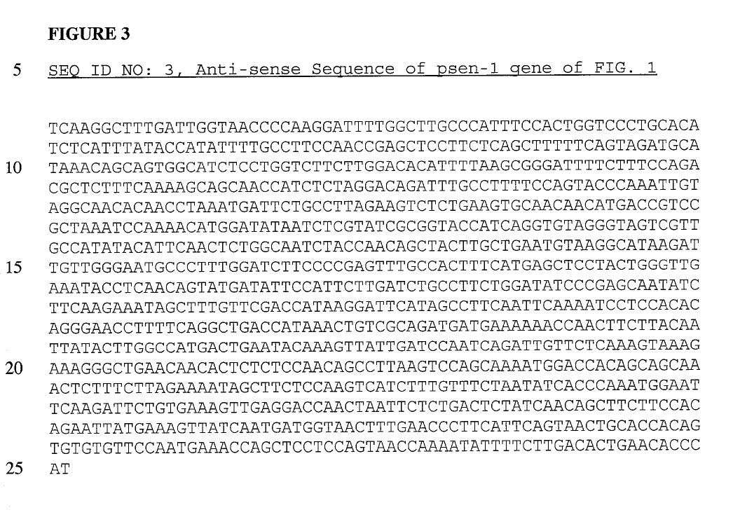

Antisense technology, however, has not been applied to potato to ameliorate or prevent cold induced sweetening in potato tubers or to alter the starch levels of crop plants. According to the present invention there is provided a nucleotide sequence which is an antisense gene encoding an antisense RNA molecule that has a nucleotide sequence complementary to a sense mRNA molecule that codes for an enzyme capable of catalyzing the interconversion of UDP-glucose and UDP-galactose. A preferred antisense gene of the present invention, shown in FIG. 3, is used to control the expression of psen-1, the nucleotide of which is shown in FIG. 1. This antisense gene is preferably under transcriptional control of a promoter and a terminator, both promoter and terminator capable of functioning in plant cells.

The antisense gene can be of any length provided that the antisense RNA molecule encoded by the antisense gene is sufficiently long to form a complex with a sense mRNA molecule encoding a potato epimerase.

For the purposes of the description of the present invention, the antisense gene can be from about 50 nucleotides in length up to a length which is equivalent to the full length of the gene. Preferably, the length of the DNA encoding the antisense RNA will be from 100 to 1500 nucleotides. The preferred gene of the present invention is a DNA which codes for an RNA having substantial sequence identity or similarity to the mRNA encoding the potato epimerase of the present invention . Thus the antisense DNA of the present invention can be selected from the group of the antisense equivalent of the psen-1 gene of the present invention or fragments thereof. The invention still further provides a nucleotide sequence which is a variant of the above disclosed antisense RNA sequences.

Alternatively, dge gene expression can be down-regulated through cosuppression. Multiple copies of a dge gene are inserted into a transgenic plant resulting in reduced expression of the dge gene.

5. Method of Increasing Gene Expression

In one embodiment, the invention provides a method for increasing expression of a dge gene, for example, psen-1 in a transformed cell or plant. The method comprises operably linking a promoter or functional promoter elements according to the present invention to the dge gene. In an alternative embodiment, the method comprises operably linking an external enhancer element to an isolated nucleic acid of this invention, which is operably linked to a promoter and/or enhancer. The resulting promoter construct increases the expression of the gene. The terms “increased” or “increasing” as used herein refer to gene expression which is elevated as compared to expression of the corresponding wild type gene that is not associated with a promoter containing an enhancer element according to the present invention.

6. Markers and Vectors

The isolated nucleic acids encoding according to the present invention are especially suitable for the construction of gene expression vectors. Methods for preparing gene expression vectors are well known to those skilled in the art. For example, the expression vector can be a plasmid into which a gene, under the control of a suitable promoter and other regulatory elements, and encoding a product of interest, has been inserted.

Optionally, a selectable marker can be associated with the construct containing the promoter or promoter elements operatively linked to the gene of the present invention, or alternatively the marker can be associated with the construct containing an enhancer element operatively linked to the promoter or which in turn are operatively linked to the gene of the present invention. As used herein, the term “marker” refers to a gene encoding a trait or a phenotype which permits the selection of, or the screening for, a plant or plant cell containing the marker. Preferably, the marker gene is an antibiotic resistance gene whereby the appropriate antibiotic can be used to select for transformed plant cells from among cells that are not transformed. Examples of suitable selectable markers include adenosine deaminase, dihydrofolate reductase, hygromycin-B-phosphotransferase, thymidine kinase, xanthine-guanine phospho-ribosyltransferase and amino-glycoside 3′-O-phosphotransferase II (kanamycin, neomycin and G418 resistance). Other suitable markers will be known to those of skill in the art. For example, screenable markers, such as the uidA, gus, luciferase or the green fluorescent protein (gfp) gene can also be used.

7. Transgenic Plants

Also disclosed are transgenic plants comprising the polynucleotide of the present invention. The isolated polynucleotide according to the present invention can be used in the same or different species from which it is derived or in which it naturally functions. The isolated polynucleotide of the present invention may be used to enhance dge gene expression in plants. Most preferably, the isolated polynucleotide according to the present invention is used for non-native gene expression in a plant. By “non-native” gene expression it is meant that a promoter and optional enhancer element operatively linked thereto controls and enables high level expression of the polynucleotide gene of the present invention that is not normally found in the host plant. The DGE enzyme is extracted from transgenic plant tissue in which the enzyme is overproduced.

The transformation of plants in accordance with the invention can be carried out in essentially any of the various ways known to those skilled in the art of plant molecular biology. (See, for example, Methods of Enzymology, Vol. 153, 1987, Wu and Grossman, Eds., Academic Press, incorporated herein by reference). As used herein, the term “transformation” refers to alteration of the genotype of a host plant by the introduction of exogenous or endogenous nucleic acid sequences.

To commence a transformation process in accordance with the present invention, it is first necessary to construct a suitable vector and properly introduce the vector into the plant cell. The details of the construction of the vectors utilized herein are known to those skilled in the art of plant genetic engineering.

For example, the isolated polynucleotide utilized in the present invention can be introduced into plant cells using Ti plasmids, root-inducing (Ri) plasmids, and plant virus vectors. For reviews of such techniques see, for example, Weissbach & Weissbach, 1988, Methods for Plant Molecular Biology, Academic Press, N.Y., Section VIII, pp. 421-463; and Grierson & Corey, 1988, Plant Molecular Biology, 2d Ed., Blackie, London, Ch. 7-9, and Florsch et al., Science 227:1229 (1985), both incorporated herein by reference.

A skilled artisan will be able to select an appropriate vector for introducing the nucleic acid sequences of the invention in a relatively intact state. Thus, any vector which will produce a plant carrying the introduced DNA sequence should be sufficient. The selection of the vector, or whether to use a vector, is typically guided by the method of transformation selected.

For example, a heterologous nucleic acid sequence can be introduced into a plant cell utilizing Agrobacterium tumefaciens containing the Ti plasmid. When using an A. tumefaciens culture as a transformation vehicle, it is most advantageous to use a non-oncogenic strain of the Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The chimeric plasmid contains at least one border region of the T-DNA region of a wild-type Ti plasmid flanking the nucleic acid to be transferred. Binary Ti plasmid systems have been shown effective to transform plant cells (De Framond, Biotechnology, 1:262, 1983; Hoekema et al., Nature 303:179 (1983). Such a binary system is preferred because it does not require integration into the Ti plasmid in Agrobacterium.

Methods involving the use of Agrobacterium include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by in situ transformation by Agrobacterium, as described by Bechtold et al., C. R. Acad Sci. Paris 316:1194 (1993). This approach is based on the vacuum infiltration of a suspension of Agrobacterium cells.

Alternatively, the isolated polynucleotide according to this invention can be introduced into a plant cell by contacting the plant cell using mechanical or chemical means. For example, nucleic acid can be mechanically transferred by direct microinjection into plant cells utilizing micropipettes. Moreover, the nucleic acid can be transferred into plant cells using polyethylene glycol which forms a precipitation complex with genetic material that is taken up by the cell.

The nucleic acid can also be introduced into plant cells by electroporation (Fromm et al., Proc. Natl. Acad. Sci., U.S.A. 82:5824 (1985), incorporated herein by reference). In this technique, plant protoplasts are electroporated in the presence of vectors or nucleic acids containing the relevant nucleic acid sequences. Electrical impulses of high field strength reversibly permeabilize plant membranes allowing the introduction of nucleic acids. Electroporated plant protoplasts reform the cell wall, divide and form a plant callus. Selection of the transformed plant cells with the transformed gene can be accomplished using phenotypic markers as described herein.

Another method for introducing nucleic acid into a plant cell is high velocity ballistic penetration by small particles with the nucleic acid to be introduced contained either within the matrix of small beads or particles, or on the surface thereof. See, for example, Klein et al., Nature 327:70 (1987). Although, typically only a single introduction of a new nucleic acid sequence is required, this method particularly provides for multiple introductions.

Cauliflower mosaic virus (CaMV) can also be used as a vector for introducing heterologous nucleic acid into plant cells (U.S. Pat. No. 4,407,956). The CaMV viral DNA genome is inserted into a parent bacterial plasmid creating a recombinant DNA molecule which can be propagated in bacteria. After cloning, the recombinant plasmid can be re-cloned and further modified by introduction of the desired nucleic acid sequence. The modified viral portion of the recombinant plasmid is then excised from the parent bacterial plasmid, and used to inoculate the plant cells or plants.

Using Agrobacterium Ti vector-mediated plant transformation methodology, all polynucleotide molecules of this invention can be inserted into plant genomes after the polynucleotide molecules have been placed between the T-DNA border repeats of suitable disarmed Ti-plasmid vectors (Deblaere, R. et al., 1987, Methods in Enzymology 153 277-292). This transformation can be carried out in a conventional manner, for example as described in EP 0116718, PCT publication WO 84/02913 and EPA 87400544.0. The polynucleotide molecule can also be in non-specific plasmid vectors which can be used for direct gene transfer (e.g. de la Pena, A., 1987, Nature, 325:274-276).

Plants transformed with psen-1 are used to regulate carbon metabolism. Preferred plants transformed according to the present invention include potatoes, Brassica, maize, rice and wheat.

8. Application in Controlled Environment Agriculture

The isolated polynucleotide of the present invention can be used in Controlled Environment Agriculture (CEA) in one embodiment of this invention. CEA employs an integrated system for commercial production of transgenic plants in a controlled environment. Plants are grown under defined environmental conditions, for example in a greenhouse, to optimize growth of the transgenic plant as well as expression of the gene encoding the protein of the present invention. In CEA, the transgenic plants can be cultivated through hydroponics or in soil-less or soil-containing media. The transgenic plants selected for heterologous protein production under the defined environmental conditions of CEA can also be grown in open field agriculture (OFA). Diverse plant species can be used including dicots and monocots.

The transgenic plants used in CEA according to the present invention are transformed with an expression vector comprising a CEA promoter operably linked to the gene of the present invention. The selected CEA promoter maximizes heterologous protein production under the corresponding environmental condition of CEA and thus can be used to alter the nutritional profile of the plant grown in CEA. If the plants are grown in high light intensities, for example, the CEA promoter would be a light-inducible promoter.

9. Production of 5′-diphospho-galactose 4-epimerase Enzyme

Host cells according to the present invention comprising a polynucleotide molecule of the present invention, or a vector containing a polynucleotide of the present invention, can be cultivated under conditions which produce a polypeptide having epimerase activity. The polypeptide can then be purified. Preferably host cells made to produce the epimerase enzyme of the present invention are transformed as described above.

Preferred host cells of the present invention are plant cells, and more preferably transgenic plants are used to produce the epimerase enzyme in commercial quantities. Preferred plants transformed according to the present invention include potatoes, Brassica, maize, rice and wheat.

Purified epimerase enzyme according to the present invention can be utilized in vitro plant material and feedstocks to convert the sugars to more usable forms. Additionally, complex molecules comprising glucose and galactose molecules, e.g. pectin which is made up of polygalacturonic acid, can first be broken down into more simple molecules by a suitable enzyme, which can then be converted into the desired sugars using the epimerase enzyme of the present invention.

Specific embodiments of the present invention are illustrated by the following non-limiting examples.

### EXAMPLE I

### Cloning of the Full Length cDNA of Potato Epimerase Gene psen-1

Referring to FIG. 1, the full-length cDNA sequence of potato psen-1 is illustrated. Healthy, fully expanded potato leaves were detached and incubated in darkness for 4 days at 30° C. Leaf materials were collected at 1, 2, 3, and 4 days of dark treatment. 1.25 μg of poly(A)⁺ mRNA isolated from each the four different treated samples was pooled and used for cDNA library construction. Double-stranded cDNA was then synthesized with a cDNA synthesis kit and inserted into the Uni-ZAP XR vector (Stratagene) after ligation of an EcoRi adaptor and a XhoI digestion, and was introduced into XL-1 blue cells (Stratagene). About 2×10⁴ primary colonies were transferred onto nylon filters. The radiolabeled cDNA probe was prepared from mixed DNA fragments of SEN1 and SEN4 clones previously isolated from Arabidopsis. Park et al. 1998, Plant Mol Biol 37:445-454; Oh et al. 1996. Plant Mol Biol 30:739-754. Plaque hybridization screening was performed by standard methods based on manufacturer's instruction in the nylon filters (Amersham-Pharmacia, Piscataway, N.J.). The psen1 clone was isolated and its cDNA insert was rescued from the Uni-ZAP XR vector using a helper phage as described by the manufacturer (Stratagene).

Sequence homology searches were performed using the nucleic acid sequence database GenBank. It was found that the sequence of the discovered fragment is homologous to the sequence of Arabidopsis thaliana uridine diphosphate glucose (UDPG) epimerase gene (86% homology at the peptide level, 71% homology at the nucleotide level). This result was further confirmed by homology searches using the amino acid sequence database Swisprot.

### EXAMPLE II

### Northern Analysis of psen-1 Expression in Potato Plants Incubated in the Dark

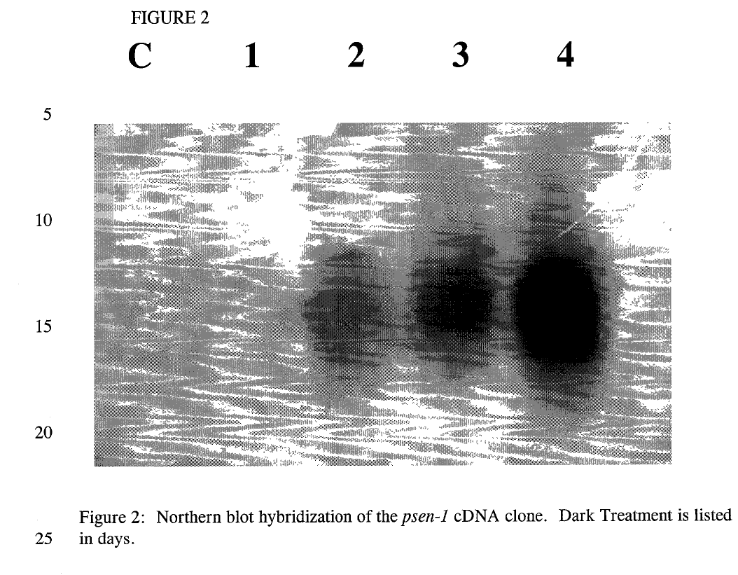

Healthy, fully expanded potato leaves were detached and incubated in 10 mM MES buffer, pH 5.5 in darkness for 4 days at 30° C. Leaf material was collected after 1, 2, 3 and 4 days of dark treatment. Twenty μg of total RNA per sample extracted from dark-treated leaves were electrophoresed on 1.3% of formaldehyde agarose gel and transferred onto a Zeta probe membrane. A (α-³²P) dCTP labeled probe was prepared from the psen1 cDNA clone. Procedures involving probe hybridization and post-hybridization were described in manufacturer instructions materials (Bio-Rad). The results of Northern blot assaying (FIG. 2) revealed that the expression of the psen1 gene is dramatically induced by dark-treatment. In FIG. 2, lanes C, 1, 2, 3, and 4 represent mRNA isolated from leaf samples before dark-treatment (C) as well as after 1, 2, 3, and 4 days of dark-treatment, respectively.

### EXAMPLE III

### Expression of psen-1 in E.coli

Psen-1 was cloned and expressed in E. coli. Primers were designed to ligate the coding sequence of the psen-1 sequence into pET15b, a His-tagged vector (Novagen). According to the method of (1996) Dorman and Benning, Arc. Bioch. Biophys. 327(1). The full-length psen-1 coding sequence from cDNA clone was fused to the His tag at the Nde1 and BamHI sites.

NcoI

```
ATACCATGGGCAGCAGCCATCATCATCATCATCACAGCAGCGGCCTGGTG
          M G S S H H H H H H S S G L V
                        NdeI XhoI BamHI-
CCGCGCGGCAGCCATATGCTCGAGGATCC
P R G S H M
```

a. Isolation of 5′-end cDNA Fragment of psen1 by PCR

Since the original psen1 cDNA clone isolated from cDNA library did not contain the 5′-end region of the epimerase coding region, this fragment was isolated by PCR using total phage lambda DNA isolated from a phage CDNA library created from senescencing potato leaves. The 3′-end anti-sense primer was designed based on the DNA sequence of the psen1 clone, 5′-TCA CCC AAA TGG AAT TCA AGA TTC TGT GAA AGT TGA GGA-3′. The 5′-end primer based on the Uni-ZAP XR vector was directly purchased from Stratagene. The PCR product was cloned into the pGEM-T vector and transferred into DH5alpha competent host cells for DNA replication. The sequences of PCR products were confirmed via an automatic DNA sequencer. The full-length cDNA of psen1 was obtained by joining the 5′-end fragment and the previously isolated cDNA clone through the EcoRI restriction site via T4 DNA ligase.

b. Preparation of a PCR Fragment for Ligation

In order to clone the full-length epimerase coding sequence into expression vector pET15b in frame, two primers (N-terminal primer and C-terminal primer) were synthesized. The N-terminal primer containing the NdeI restriction enzyme site (CATATG) was: 5′-GGA ATT CATATG GGT GTT CAG TGT CAA GAA AAT ATT TTG GT-3′. The C-terminal primer containing BamHI restriction enzyme site (GGATCC) was: 5′-GGA ATT GGATCC TTA TCA AGG CTT TGA TTG GTA ACC CCA AG-3′. Thirty cycles of PCR reaction conditions were 95° C. for 1 min, 58° C. for 2 min, and 72° C. for 2 min. The PCR product and pET15b vector DNA were digested with NdeI and BamHI restriction enzymes. The DNA fragment of NdeI and BamHI from PCR products was mixed with pET15b vector DNA digested with NdeI and BamiHI enzymes in a 5:1 ratio and ligated at 16° C. overnight. The ligation mixture was transferred into the DH5alpha host cells for DNA duplication.

c. Mini-Scale Plasmid DNA Preparation for Insertion Screening

The plasmid DNA was prepared in mini-scale. The insertion of psen1 gene was confirmed by enzyme restriction analysis. When the plasmid DNA was digested with NdeI and BamHI enzymes, the full-length coding sequence was excised, resulting in an approximately 1.2 kb DNA fragment containing the complete coding sequence. The sequence of plasmid DNA was further confirmed via automated DNA sequencing.

d. Transformation of Expression Cell Line BL21 (DE3)

BL21 (DE3) host cells containing pET15b expression vector were used for epimerase expression. The transformation procedure was described in instructions from the manufacturer (Novagen).

e. Small Scale Expression Cultures

Epimerase expression was induced in three ml aliquots from each transformed BL21 line were induced using IPTG as per manufacturers instructions (Novagen). Each 3 ml sample was then divided in half and placed into two separate microcentrifuge tubes and centrifuged to obtain cell pellets. Protein extraction was then completed on each pellet fraction following the instruction of manufacturer (Novagen). One pellet extract was used for SDS-PAGE and other for activity testing.

f. Screen Cultures by SDS-PAGE for Protein Production

100 μl of cell extract containing approximately 20 μg protein per sample was used for SDS-PAGE. The protein loading buffer containing 50 mM Tris-HCl (pH 6.8), 2.5% SDS, 7% glycerol, 1 M beta-mercaptoethanol, and trace amounts of bromophenol blue. The detailed procedure employed has been described previously (Dai et al. 2000. Transgenic Res 9:43-54).

g. Screening Cultures by Epimerase Activity Assay

Cell pellets were treated with 50 μl Lysis Buffer (50 mM Tris, pH 8.0, 100 mM NaCl, 1 mM EDTA, 0.3 mg/ml lysozyme, 7 μg/ml DNAse I). The cell mixtures were shaken at room temperature for 20 minutes and centrifuged at 20,000 g for 10 minutes at 4° C. The clear supernatant was transferred into a new tube. About 10 to 50 μl of supernatant was used for activity measurement. Enzymatic Assay of Uridine 5′-Diphosphogalactose 4-Epimerase (Fukasawa et al. 1980. J Biol Sci 255:2705-2707)(incorporated herein by reference in its entirety) was assayed as follows.

The following reagents were mixed at 25° C. in plastic cuvettes: 2.38 mL deionized water, 0.30 mL 1M glycine buffer (pH 8.8), 0.06 mL 5 mM uridine 5′-diphosphogalactose, 0.06 mL 50 mM [symbol] -nicotinamide adenine dinucleotide and 0.10 mL 2 units/mL uridine 5′-diphosphoglucose dehydrogenase. An enzyme diluent (0.10 mL of 100 mM citrate at pH 7.0) and enzyme solution (E. coli lysate) were added to reagent solutions in the BLANK cuvette and SAMPLE cuvettes, respectively. Standard solution of UDP-galactose epimerase at a concentration of 0.05 U/mL was added to reagent solution in the POSITIVE CONTROL cuvette. Cuvettes were immediately mixed by inversion and change in absorbance at 340 nm was measured spectrophotometrically. Enzyme activity (in U/mg total protein) was measured as the absorbance increase (over 5 minutes) denoting the formation of UDP-glucose as compared to that of the BLANK solution. Results for 4 separate E. coli lysate samples, a negative control and a positive control are reported in the table below:

TABLE 1
UDP-Galactose Epimerase Activity in E. coli lysate samples

Enzymatic Activity

| | (Units/mL | (Units/mg total |
|---|---|---|
| Designation | solution) | protein)* |
| Positive Control | 0.005 | — |
| Negative Control (E. | −0.001 | −0.003 |
| coli strain BL21 | | |
| lysate) | | |
| Sample #1 | 0.024 | 0.149 |
| Sample #2 | 0.034 | 0.418 |
| Sample #3 | 0.025 | 0.108 |
| Average of samples | 0.028 | 0.225 |
| Standard deviation in | 0.005 | 0.169 |
| samples | | |

*There appears to be a substantial degree of error in the BioRad protein determination in the transformed E. coli samples

### EXAMPLE IV

### Expression of psen-1 in Transgenic Potato

This example describes gene cloning and stable transformation of the expression of the potato UDP-galactose epimerase gene. This system may be used to increase or control the production of functional epimerase in planta leading to applications related to agronomic benefit, in which the epimerase remains in crops as well as applications related to bioprocessing, in which the epimerase would be extracted from specific plant portions and used in an in vitro process setting. Crop trait modification via epimerase overexpression could lead to better utilization of carbon energy stores under specific environmental conditions, including stress response conditions, as the epimerase enzyme could help increase glucose levels in planta. In a process setting, epimerase may be able to enrich feedstocks of glucose and other easily convertible, metabolizable sugars.

Bacterium Strain and Plant Materials

Escherichia coli DH5α is used as the host for routine cloning experiments. The A. tumefaciens strain PC2760 is chosen as the host for the binary vectors. N. tabacum cell suspension culture designated NT1 is used for cell culture experiments. Solanum tuberosum cv. Desiree is used as the host for Agrobacterium-mediated transformation and for genomic DNA isolation.

Stable Transformation for Epimerase Expression

The transcriptional fusion construct rbcs-3c::psen1 is prepared by inserting the tomato ribulose bis-phosphate carboxylase small subunit (RbcS-3C) promoter: :psen1 gene fragment as a cassette into a plant expression binary vector. The chimeric promoter/psen1 construct is transferred into tobacco cell culture and potato plants via Agrobacterium transformation using the method described in Dai et al., 2000, Mol Breed 6: 227-285. At least 100 independently transformed calli and 30 independently transformed plants are regenerated. Epimerase levels in transgenic calli are measured under normal growth conditions, using the methods described in Example III. In addition, epimerase levels in transgenic primary plant transformants are measured under normal growth and abscission conditions.

For protein production applications (i.e., in vitro use of the epimerase enzyme), CEA could comprise a transgenic plant transformed with an expression vector comprising a CEA promoter, such as RbcS-3C, operably linked to a gene encoding the heterologous protein of interest. Preferably, the plant used in this protein production system is selected because under conditions of CEA it produces (1) rapid and efficient growth of harvested plant biomass containing the heterologous protein; (2) large amounts of heterologous protein in the harvested plant biomass; and (3) a plant tissue extract wherein the heterologous protein is stable.

### Example V

### Regulation of psen1 Expression

In order to regulate psen1 gene expression by introducing antisense genes, 1.1 kb psen1 cDNA in reverse orientation (FIG. 3) is operably linked to the Cauliflower Mosaic Virus (CaMV) 35S promoter with the B-domain enhancer or the tomato RbcS-3 promoter and terminated by the transcriptional terminator of the nopaline synthase gene. These transgene expression constructs are introduced into transgenic potato plants via Agrobacterium-mediated transformation. The detailed method was described in previous reports (Dai et al., 2000, Mol Breed 6: 227-285). Preferably, more than 50 individual transformant plants are analyzed for epimerase activity as described above in example III.

We claim:

1. An isolated polynucleotide molecule comprising a gene coding for the enzyme 5′-diphospho galactose 4-epimerase (DGE) selected from the group consisting of:
(a) the polynucleotide molecule shown in FIG. 1;
(b) a polynucleotide molecule coding for a protein having the amino acid sequence shown in FIG. 1; and
(c) a polynucleotide that hybridizes under stringent conditions to the polynucleotide sequence shown in FIG. 1 and codes for an enzyme having 5′-diphospho galactose 4-epimerase (DGE) activity.

2. The isolated polynucleotide according to claim 1 wherein the gene coding for DGE is operably linked to a heterologous promoter.

3. A vector comprising a polynucleotide molecule according to claim 1.

4. A host cell comprising the vector of claim 2.

5. The host cell of claim 4, wherein the host cell is a plant cell.

6. A plant comprising a plant cell according to claim 5.

7. The plant according to claim 6, wherein the plant is selected from the group of plants consisting of potatoes, Brassica, maize, rice and wheat.

8. A method for producing a transgenic plant with reduced DGE enzyme activity, comprising the steps of: a) preparing an antisense gene corresponding to the dge gene; b) transforming a plant with the antisense gene; and c) identifying a fertile transgenic plant comprising the antisense gene exhibiting DGE enzyme activity.

9. A method for producing DGE comprising the steps of (a) transforming a plant with a polynucleotide molecule comprising a gene coding for DGE and (b) recovering DGE from the transformed plant.
